(19) United States
(12) Patent Application Publication
(10) Pub. No.: US 2005/0214916 A1
(43) Pub. Date: Sep. 29, 2005

(54) Biological process for synthesis of oxide nanoparticles

(75) Inventors: Ahmad Absar, Pune (IN); Murali Sastry, Pune (IN); Debabrata Rautaray, Pune (IN)

Correspondence Address: DARBY & DARBY P.C., P. O. BOX 5257, NEW YORK, NY, 10150-5257, US

(73) Assignee: Council of Scientific and Industrial Research, New Delhi (IN)

(21) Appl. No.: 10/810,338

(22) Filed: Mar. 26, 2004

(51) Int. Cl. C12P 1/00; C12N 1/16

(52) U.S. Cl. 435/168; 435/254.3

(57) ABSTRACT

The present invention provides a biological process for the synthesis of oxide nanoparticles by simple exposure of suitable aqueous metal ions to a hydrolyzing fungus. The method specifically comprises reacting of a suitable electrolyte solution with a hydrolyzing wet fungus.

### FIELD OF THE INVENTION

The present invention relates to a biological process for the synthesis of oxide nanoparticles by simple exposure of suitable aqueous metal ions to a hydrolyzing fungus. More particularly the present invention relates to a biological process for the synthesis of oxide nanoparticles by the reaction of suitable electrolyte solution with a hydrolyzing wet fungus. The present invention also relates to a biological process for the synthesis of oxide nanoparticles controlled by the proteins secreted- by the respective fungus, which are responsible for the size and shape control of the desire oxide nanoparticles after separating the biomass. The invention also provides a method for producing shape, size and polymorph controlled oxide nanoparticles such as titanium oxide (TiO₂) zirconium oxide (ZrO₂), silicon oxide (SiO₂), zinc oxide (ZnO) using naturally occurring biomaterials such as wet fungus or their extract.

### BACKGROUND OF THE INVENTION

Biological and materials synthesis and transformation is a core industry in the world economy. Various techniques have been developed for large-scale generation of inorganic materials of controllable structure and size based on either physical or chemical principles. Numerous substances are synthesized using processes that require non-ambient temperature and/or non-ambient pressure that require capital-intensive equipment. Methods that can produce useful chemicals and materials at conditions closer to ambient conditions that use simple equipment are economically, ecologically and environmentally more desirable.

Significant research efforts have been devoted for nanostructure synthesis as a means to achieved materials having commercial in areas as diverse ceramics, electronics, pigments, cosmetics industries (Mann et al. Nature, 1996, 382, 313, Matijevic et al. Curt Opin Colloid Interface Sci., 1996, 1, 176). To date zirconia-based ceramic alloys have been demonstrated to be the strongest and toughest oxide ceramics yet produced ZrO₂—toughened ceramics, in which the toughening process is based on the ZrO₂ transformation, have been used in many materials systems. Pure ZrO₂ at ambient temperature is polymorphic, exhibiting cubic (C) (fluorite) structure (Fm3m) at higher temperature (>2370° C.), tetragonal structure (P4₂/nmc) at intermediate temperatures (1200°-2370° C.) and monoclinic symmetry (P2₁/c) at low temperature (<950° C.) (Monte et al., J. Am. Ceram Soc. 2000. 83, 328). The most dramatic increase in its industrial applicability has been brought about by the discovery that the t→m transformation on cooling below 950° C. can be controlled by suitable material processing to become the source of transformation plasticity and transformation toughening in tailored, two-phase microstructures (Garvie et al, Nature 1975, 258, 258, 703). Transformation toughening of ZrO₂ was first reported in a paper entitled “Ceramic Steel” (Gravie et al, Nature 1975, 258, 703). Kisi and Howard (1998 in Key Engineering Materials. Vols. 153, pp 1-36) provide a recent review if the phases observed in ZrO₂ and its alloys. Various efforts have been made in the fabrication of highly crystalline gallium oxide nano tubes, wire and brushes using molten gallium and microwave plasma treatment (Sharma et al. J. Am. Chem. Soc., 2002, 124, 12288), hierarchical ZnO nanostructure by vapor transport and condensation technique (Lao et al. Ano Lett., 2002, 2, 1287).

Stober et al have synthesized silica nanoparticles in the size range of 200-2000 nm. Silica nanospheres were produced by reaction between tetraesters of silicic acid (tetraalkyl silicates) with ammonia as a catalyst in presence of alcohol (Stober, Fink and Bohn, J. Colloid Interface Sci. 1968, 26, 62). New structures (hexagons) of amorphous silica were synthesized in vitro by incubating 50 mg/ml poly-L-lysine (PLL) in 1M pre-hydrolysed tetramethoxysilane (Patwardhan et al, Chem. Commun. 2003, 1122).

Titanium, zirconium and hafnium dioxides were synthesized by using self-assembled monolayers of organosilicon hydrides (RSiH₃). The reactions of alkyl-, fluoroalkyl- and w-alkenyl-silanes and alpha-w-bis-hydridosilanes with nanoporous TiO₂ (anatase), ZrO₂ (monoclinic) and HfO₂ (monoclinic) powders were synthesized (Fadeev et al Langmuir 2002, 18, 7521). Lin Shi et al have synthesized nanosized TiO₂ in the pore channels of a silica-based mesoporous thin films (Lin Shi et al, Advanced Materials 2002, 14, 830). Amorphous titanium dioxides were coated with colloidal polystyrene spheres. The core-shell particles can be turned into spherical hollow titania shells by dissolution of the polystyrene cores in suspension or by calcinations of the dried particles in a furnace (Imhof et al, Langmuir 2001, 17, 3579). It is know that the presence of impurity in semiconducting nanoparticles results in increasing photodegradation rate and impurities play an important role in modulating the properties of semiconducting nanoparticles, which leads to the concept of introduction of dopant to optimize the semiconducting behavior or new crystalline phase. Film nanostructure has been optimized for maximum photodegradation efficiency by controlling the original reverse micellar composition, the ripening of the particles and the thickness of the films. Films doped with silver ions, incorporated through the reverse micellar route, are more efficient photocatalysts than pure titanium films and become even more efficient when they are treated with UV irradiation. Film doped with ruthenium ions are less efficient for photocatalysis but when they are treated with UV radiation they also become more efficient photocatalysts than pure titania films (Stathatos et al, Langmuir 2001, 17, 5025). Iron doped titania photocatalysts with different iron contents were prepared by using a solgel method in acidic media (Wang et al, J. Phys. Chem B 2001, 105, 9692). Braun et al have reported a stable suspension of fluorescent erbium-doped titania nanoparticles and their assembly into thin films and photonic crystals (Braun et al, Chem Mater. 2003, 15, 1256). Crystalline TiO₂ film has been deposited on several substrates (glass, F-doped, SnO₂-covered glass and silicon wafers) by a drain-coating method from a colloidal anatase aqueous solution at low temperature (Peiro et al, Chem. Mater. 2001, 13, 2567). Butanediol sol-gel synthesis for Fe doped titania is also described which allows control of the material properties on atomic, mesoscopic, and macroscopic scales with respect to structure-morphology, property and relationship (Zhang et al Chem. Mater. 2003, 15, 4028). Zirconia nanoparticles doped with up to 50 mol % Al₂O₃ were prepared by chemical vapor synthesis (CVS) (Srdic et al, Chem. Mater. 2003, 15, 2668). Ultrasound radiation was used to prepare Eu₂O₃ in zirconia and yttrium-stabilized (YSZ) nanoparticles (Gedanken et al, Phys. Chem B. 2000, 104, 7057). Erbium doped ZrO₂ nanoparticles are prepared by a sol-emulsion-gel technique. The effects of the Er³⁺ concentration and different co-dopants (Yb³⁺ and Y³⁺) in ZrO² matrix on the up converted emission are observed (Prasad et al, J. Phys. Chem. B. 2002, 106, 1909).

U.S. Pat. No. 6,136,186 describes a method and apparatus for mineralizing organic contaminants in water or air by a photochemical oxidation in a two-phase or three-phase boundary system formed in the pores of a TiO₂ membrane in a photocatalytic reactor. In the three-phase system, gaseous (liquid) oxidant, liquid (gaseous) contaminant, and solid semiconductor photocatalyst meet and engage in an efficient oxidation reaction. The porous membrane has pores, which have a region wherein the meniscus of the liquid varies from the molecular diameter of water to that of a capillary tube resulting in a diffusion layer that is several orders of magnitude smaller than the closest known reactors. The photocatalytic reactor operates effectively at ambient temperature and low pressures.

U.S. Pat. No. 6,586,095 discloses a method for preparing a plurality of semiconductor oxide nanostructures that have a substantially rectangular cross-section from an oxide powder. A representative method includes heating the oxide power to an evaporation temperature of the oxide powder for about 1 hour to about 3 hours at about 200 torr to about 400 torr in a atmosphere comprising argon; evaporating the oxide power, and forming the plurality of semiconductor oxide nanostructure.

The prior art methods for the growth of various oxide nanoparticles teach growth of a wide variety of such particles together with control over their crystal size, shape and morphology but have certain limitations.

The major drawbacks of the prior art processes compare to the present invention are:
1. The methods are not environmentally friendly and simple.
2. Large-scale synthesis is not possible.
3. Not Cost effective/Economical system for the industry.
4. Uniform size control is tough
5. Complex experimental conditions
6. Require more maneuvering
7. Not a robust system.
8. Stability of the system is low
9. There is an upper limit to scaling in terms of mass production
10. Possibility of contamination is high if proper care is not taken.

It is therefore important to provide an efficient and simple method for the production of oxide nanoparticles in order to reduce the complexity of the above mentioned processes and to enhance the large scale production of oxide nanoparticles.

### OBJECTS OF THE INVENTION

The main object of the invention is to provide a biological process for the synthesis of semiconducting oxide nanoparticles, which are environmental friendly.

It is another object of the invention to provide a process for the preparation of shape, size and polymorph controlled synthesis of oxide nanoparticles that are user friendly.

It is yet another object of the invention to provide an economic and efficient process for the synthesis of shape, size and polymorph controlled oxide nanoparticles.

These and other objects of the invention are achieved by the process of the invention which uses a biological method for the synthesis of shape, size and polymorph controlled oxide nanoparticles.

### SUMMARY OF THE INVENTION

Accordingly, the present invention provides a biological process for the synthesis of shape, size and polymorph controlled oxide nanoparticles, which comprises incubating a wet fungus or fungal extract with an aqueous solution of a metal salt to obtain a biomass, separating the biomass and filtering the oxide nanoparticles therefrom.

In one embodiment of the invention, the incubation of the wet fungus/fungal extract with the metal salt solution is carried out at a temperature in the range of 15 to 40° C. and for a period in the range of 1 to 3 days.

In another embodiment of the invention, the filtration is carried out using a minimum 1 mcron pore size filter to obtain the oxide nanoparticles.

In another embodiment of the invention, the metal salt is selected from the group consisting of chlorides, nitrates, oxalates and sulfates.

In another embodiment of the invention, the fungus is used in whole cell form.

In another embodiment of the invention, the temperature for incubation is in the range of 23-33° C., preferably 25-29° C.

In another embodiment of the invention, the concentration of the metal salt in the solution is not less than 1 mM.

In another embodiment of the invention, the fungus/fungal extract is used in an amount of 10 to 60 mgs.

In yet another embodiment of the invention, the fungi used are selected from the group consisting of Fusarium sp., Trichothecium sp., Verticillium sp., Chloridium sp., Aspergillus sp., Cephaliophora sp., Fusarium oxysporum and Helicostylum sp.

In another embodiment of the invention the metal comprises a metal from the transition metal group.

In yet another embodiment of the invention, the metal is selected from the group consisting of Ti, Zr, Si and Zn.

### DETAILED DESCRIPTION OF THE INVENTION

The present invention provides a biological process for the synthesis of oxide nanoparticles by simple exposure of suitable aqueous metal ions to a hydrolyzing fungus. The method specifically comprises reacting of a suitable electrolyte solution with a hydrolyzing wet fungus. The synthesis of oxide nanoparticles is controlled by the proteins secreted by the respective fungus, which enable the size and shape control of the desired oxide nanoparticles after separation of the biomass. Examples of the metal oxide nanoparticles that may be obtained with shape, size and polymorph control are titanium oxide (TiO₂) zirconium oxide (ZrO₂), silicon oxide (SiO₂), zinc oxide (ZnO). The fungus is used in either whole cell form or wet fungal extract form.

The shape, size and polymorph controlled particles formed by the process of the invention can be used in numerous technological and medical applications, e.g., electronics, as advanced ceramics, catalysts, sensors, semiconductors, pigments, can be used in cosmetic and medical industries and many others. All publications and patents mentioned herein are incorporated by references. While this invention has been described in relation to certain preferred embodiments thereof and many details have been set forth for purpose of illustration, it will be apparent to those skilled in the art that additional embodiments are possible without departing from the spirit and scope of the invention

The incubation of the wet fungus/fungal extract with the metal salt solution is carried out at ambient, preferably at a temperature of 15 to 40° C. and for a period of 1 to 3 days. The temperature for incubation is more preferably in the range of 23-33° C., and more particularly 25-29° C. The biomass is filtered using a minimum 1 micron pore size filter to obtain oxide nanoparticles. The metal is from the transition metal group. Examples of metals whose oxides are obtained in the form of nanoparticles are Ti, Zr, Si and Zn. The metal salt solution can be a chloride, nitrate, oxalate or a sulfate. The concentration of the metal salt in the solution is not less than 1 mM. The fungus//fungal extract is used in an amount of 10 to 60 mgs. The fungus used is a naturally occurring fungi selected from Fusarium sp., Trichothecium sp., Verticillium sp., Chloridium sp., Aspergillus sp., Cephaliophora sp., Fusarium oxysporum and Helicostylum sp.

The process of the invention comprises a simple and efficient biological method which comprises using microorganisms for synthesis of semiconducting oxide nanoparticles. Suitable metal salts are exposed to various hydrolyzing fungi or their extract resulting in controlled size, shape and polymorphs of the desired oxide nanoparticles.

Some example of the nanoparticles obtained are given in the table below

| | Metal | | | | |
|---|---|---|---|---|---|
| Fungus | salt | Oxide | Polymorph | Shape | Size (nm) |
| Fusarium | K₂TiF₆ | TiO₂ | Brookite | Spherical/ | 10 nm to 50 nm |
| Oxysporium | | | | square | |
| Fusarium | K₂ZrF₆ | ZrO₂ | Baddelyte | Spherical | 2 nm to 20 nm |
| Oxysporium | | | (Monoclinic) | | |
| Fusarium | K₂SiF₆ | SiO₂ | Silica (crystalline/ | Spherical/ | 20 nm to 200 nm |
| Oxysporium | | | amorphous) | circular | |
| Fusarium Sp. | K₂TiF₆ | TiO₂ | Mixture (Brookite/ | Spherical | 20 nm to 50 nm |
| | | | Rutile) | square | |
| Fusarium Sp. | K₂ZrF6 | ZrO₂ | Baddelyte | Spherical | 2 nm to 20 nm |
| | | | (Monoclinic) | | |
| Fusarium Sp. | K₂SIF₆ | SiO₂ | Silica (crystalline/ | Spherical/ | 20 nm to 200 nm |
| | | | amorphous) | circular | |
| Trichothecium Sp. | K₂TiF₆ | TiO₂ | Brookite | Spherical/ | 20 nm to 50 nm |
| | | | | square | |

The following examples are illustrative and should not be construed to limit scope of the invention in any manner.

### EXAMPLE 1

This example illustrates the synthesis of titanium oxide (TiO₂) nanoparticles using a hydrolyzing fungus (Fusarium oxysporum) which was maintained on potato dextrose-agar (PDA) slants. Stock culture was maintained by sub-culturing at monthly intervals. After growing at pH 7 and 27° C. for four days, the slants were preserved at 15° C. From an actively growing stock culture, subculture was made on fresh slant and after four days of incubation at pH 7 and 27° C. was used as the starting material for fermentation experiment. The fungus was grown in 500 ml Erlenmeyer flask containing 100 ml malt extract-glucose yeast extract-peptone (MGYP) medium which is composed of malt extract (0.3%), glucose (1%) yeast extract (0.3%) and peptone (0.5%). After adjusting the pH of the medium to 7, the culture was grown with continuous shaking on a rotary shaker (200 rpm) at 27° C. for 96 hours. After 96 hours of fermentation, mycelia were separated from the culture broth by centrifugation (500 rpm) at 20° C. for 20 minutes and then the mycelia were washed thrice with sterile distilled water under sterile conditions. The harvested mycelia mass (20 g wet wt. of mycelia) was then re-suspended in 100 ml of 10⁻³ M K₂TiF₆ solution in 500 ml Erlenmeyer flasks. The whole mixture was put into a shaker at 27° C. (200 rpm) and the reaction carried out for a period of 48 hours. The bio-transformation were collected by separating the fungal mycelia or aqueous extract time to time by filtration from the solution containing the mycelia inside the inoculation chamber under stirrer condition and was monitored by periodic sampling of aliquots (10 ml) of the aqueous solution and was characterized by Fourier-transform infrared spectroscopy (FTIR), X-ray diffraction (XRD), Scanning electron microscopy (SEM) and Energy dispersive analysis of X-ray (EDAX). The nanoparticles formed were dried and the powder formed was calcined at 100, 300, 600 and 800° C. for the phase transformation into other polymorphic form (crystalline phase) and were further characterized.

### EXAMPLE 2

This example illustrates the synthesis of titanium oxide (TiO₂) nanoparticles using a hydrolyzing fungus, (Fusarium oxysporum) (Fungus culturing details—see Example 1). Harvested mycelia mass (20 g wet wt. of mycelia) was then resuspended in 100 ml of 10⁻³M titanium oxalate [Ti (C₂O₄)₂] solution in 500 ml Erlenmeyer flasks. The whole mixture was put in to a shaker at 27° C. (200 rpm) and the reaction carried out for a period of 48 hours. The bio transformation were collected by separating the fungal mycelia or aqueous extract time to time by filtration from the solution containing the mycelia inside the inoculation chamber under stirrer condition and was monitored by periodic sampling of aliquots (10 ml) of the aqueous solution and was characterized. The nanoparticles formed were dried and the powder formed was calcined at 100, 300, 600 and 800° C. for the phase transformation into other polymorphic form (crystalline phase) and were further characterized.

### EXAMPLE3

This example illustrates the synthesis of zirconium oxide (ZrO₂) nanoparticles using a hydrolyzing fungus, (Fusarium oxysporum) (Fungus culturing details—see Example 1). The harvested mycelia mass (20 g wet wt. Of mycelia) was then resuspended in 100 ml of 10⁻³M K₂ ZrF₆ solution in 500 ml Erlenmeyer flasks. The whole mixture was put in to a shaker at 27° C. (200 rpm) and the reaction carried out for a period of 48 hours. The bio transformation were collected by separating the fungal mycelia or aqueous extract time to time by filtration from the solution containing the mycelia inside the inoculation chamber under stirrer condition and was monitored by periodic sampling of aliquots (10 ml) of the aqueous solution and was characterized. The nanoparticles formed were dried and the powder formed was calcined at 100, 300, 600 and 800° C. for the phase transformation into other polymorphic form (crystalline phase) and were further characterized.

### EXAMPLE 4

This example illustrates the synthesis of zirconium oxide (ZrO₂) nanoparticles using a hydrolyzing fungus, (Fusarium oxysporum) (Fungus culturing details—see Example 1). The harvested mycelia mass (20 g wet wt. Of mycelia) was then resuspended in 100 ml of 10⁻³M Zirconium oxychloride (ZrOl₂) solution in 500 ml Erlenmeyer flasks. The whole mixture was put in to a shaker at 27° C. (200 rpm) and the reaction carried out for a period of 48 hours. The bio transformation were collected by separating the fungal mycelia or aqueous extract time to time by filtration from the solution containing the mycelia inside the inoculation chamber under stirrer condition and was monitored by periodic sampling of aliquots (10 ml) of the aqueous solution and was characterized. The nanoparticles formed were dried and the powder formed was calcined at 100, 300, 600 and 800° C. for the phase transformation into other polymorphic form (crystalline phase) and were further characterized.

### EXAMPLE 5

This example illustrates the synthesis of silicon oxide (SiO₂) nanoparticles using a hydrolyzing fungus, (Fusarium oxysporum) (Fungus culturing details—see Example 1). The harvested mycelia mass (20 g wet wt. Of mycelia) was then resuspended in 100 ml of 10⁻³M K₂SiF₆ solution in 500 ml Erlenmeyer flasks. The whole mixture was put in to a shaker at 27° C. (200 rpm) and the reaction carried out for a period of 48 hours. The bio transformation were collected by separating the fungal mycelia or aqueous extract time to time by filtration from the solution containing the mycelia inside the inoculation chamber under stirrer condition and was monitored by periodic sampling of aliquots (10 ml) of the aqueous solution and was characterized. The nanoparticles formed were dried and the powder formed was calcined at 100, 300, 600 and 800° C. for the phase transformation into other polymorphic form (crystalline phase) and were further characterized.

### EXAMPLE 6

This example illustrates the synthesis of silica oxide (SiO₂) nanoparticles using a hydrolyzing fungus, (Fusarium sp. (Fungus culturing details—see Example 1). The harvested mycelia mass (20 g wet wt. Of mycelia) was then resuspended in 100 ml of 10⁻³M tetraethyl ortho silicate (TEOS) solution in 500 ml Erlenmeyer flasks. The whole mixture was put into a shaker at 27° C. (200 rpm) and the reaction carried out for a period of 48 hours. The bio transformation were collected by separating the fungal mycelia or aqueous extract time to time by filtration from the solution containing the mycelia inside the inoculation chamber under stirrer condition and was monitored by periodic sampling of aliquots (10 ml) of the aqueous solution and was characterized. The nanoparticles formed were dried and the powder formed was calcined at 100, 300, 600 and 800° C. for the phase transformation into other polymorphic form (crystalline phase) and were further characterized.

### EXAMPLE 7

This example illustrates the synthesis of silica oxide (SiO₂) nanoparticles using a hydrolyzing fungus, (Fusarium sp.) (Fungus culturing details—see Example 1). The harvested mycelia mass (20 g wet wt. Of mycelia) was then resuspended in 100 ml of 10⁻³M tetraethyl ortho silicate (TMOS) solution in 500 ml Erlenmeyer flasks. The whole mixture was put in to a shaker at 27° C. (200 rpm) and the reaction carried out for a period of 48 hours. The bio transformation were collected by separating the fungal mycelia or aqueous extract time to time by filtration from the solution containing the mycelia inside the inoculation chamber under stirrer condition and was monitored by periodic sampling of aliquots (10 ml) of the aqueous solution and was characterized. The nanoparticles formed were dried and the powder formed was calcined at 100, 300, 600 and 800° C. for the phase transformation into other polymorphic form (crystalline phase) and were further characterized.

### EXAMPLE 8

This example illustrates the synthesis of silica oxide (SiO₂) nanoparticles using a hydrolyzing fungus, (Fusarium sp.) (Fungus culturing details—see Example 1). The harvested mycelia mass (20 g wet wt. Of mycelia) was then resuspended in 100 ml of 10⁻³M silicic acid (H₂SiO₃) solution in 500 ml Erlenmeyer flasks. The whole mixture was put into a shaker at 27° C. (200 rpm) and the reaction carried out for a period of 48 hours. The bio transformation were collected by separating the fungal mycelia or aqueous extract time to time by filtration from the solution containing the mycelia inside the inoculation chamber under stirrer condition and was monitored by periodic sampling of aliquots (10 ml) of the aqueous solution and was characterized. The nanoparticles formed were dried and the powder formed was calcined at 100, 300, 600 and 800° C. for the phase transformation into other polymorphic form (crystalline phase) and were further characterized.

### EXAMPLE 9

This example illustrates the synthesis of silica oxide (SiO₂) nanoparticles using a hydrolyzing fungus, (Fusarium sp.) (Fungus culturing details—see Example 1). The harvested mycelia mass (20 g wet wt. Of mycelia) was then resuspended in 100 ml of 10⁻³ M silicic acid (H₂SiO₃ 50 ml) and of 10 M (50 ml) tetraethyl ortho silicate (TEOS) solution in 500 ml Erlenmeyer flasks. The whole mixture was put into a shaker at 27° C. (200 rpm) and the reaction carried out for a period of 48 hours. The bio transformation were collected by separating the fungal mycelia or aqueous extract time to time by filtration from the solution containing the mycelia inside the inoculation chamber under stirrer condition and was monitored by periodic sampling of aliquots (10 ml) of the aqueous solution and was characterized. The nanoparticles formed were dried and the powder formed was calcined at 100, 300, 600 and 800° C. for the phase transformation into other polymorphic form (crystalline phase) and were further characterized.

### EXAMPLE 10

This example illustrates synthesis of titanium oxide (TiO₂) nanoparticles using a hydrolyzing fungus, (Fusarium sp.) (Fungus culturing details—see Example 1). Harvested mycelia mass (20 g wet wt. of mycelia) was then resuspended in 100 ml of 10⁻³M K₂TiF₆ (50 ml) and 10⁻³ M (50 ml) of titanium oxalate [Ti(C₂O₄)₂] solution in 500 ml Erlenmeyer flasks. The whole mixture was put into a shaker at 27° C. (200 rpm) and the reaction carried out for a period of 48 hours. The bio transformation were collected by separating the fungal mycelia or aqueous extract time to time by filtration from the solution containing the mycelia inside the inoculation chamber under stirrer condition and was monitored by periodic sampling of aliquots (10 ml) of the aqueous solution and was characterized. The nanoparticles formed were dried and the powder formed was calcined at 100, 300, 600 and 800° C. for phase transformation into other polymorphic form (crystalline phase) and were further characterized.

### EXAMPLE 11

This example illustrates the synthesis of zirconium oxide (ZrO₂) nanoparticles using a hydrolyzing fungus, (Fusarium sp.) (Fungus culturing details—see Example 1). The harvested mycelia mass (20 g wet wt. Of mycelia) was then resuspended in 100 ml of 10⁻³M K₂ ZrF₆ (50 ml) and 10⁻³ M (50 ml) of zirconium oxychloride (ZrOCl₂) solution in 500 ml Erlenmeyer flasks. The whole mixture was put into a shaker at 27° C. (200 rpm) and the reaction carried out for a period of 48 hours. The bio transformation were collected by separating the fungal mycelia or aqueous extract time to time by filtration from the solution containing the mycelia inside the inoculation chamber under stirrer condition and was monitored by periodic sampling of aliquots (10 ml) of the aqueous solution and was characterized. The nanoparticles formed were dried and the powder formed was calcined at 100, 300, 600 and 800° C. for the phase transformation into other polymorphic form (crystalline phase) and were further characterized.

Advantages of the process claimed in the present invention are:
1. The main advantage of the present invention is the use of biological process for the synthesis of shape, size and polymorph controlled oxide nanoparticles, which is simple and efficient.
2. The invention uses naturally occurring fungi under aqueous medium.
3. The oxide nanoparticles formed are highly controlled in shape and uniform in size.
4. The oxide nanparticles formed are quite stable in the aqueous solution.
5. Different polymorphs (crystalline phase) of the suitable oxide nanoparticles can be achieved by using suitable fungal mass in the form of wet solid mass, whole cell or fungal extract.
6. Large scale synthesis is possible under ambient conditions.
7. The process is cost effective and economical and therefore useful in industry.
8. The method is also environmentally friendly and simple.

1. A biological process for the synthesis of shape, size and polymorph controlled oxide nanoparticles, which comprises incubating a wet fungus or fungal extract with an aqueous solution of a metal salt to obtain a biomass, separating the biomass and filtering the oxide nanoparticles therefrom.

2. A process as claimed in claim 1 wherein the incubation of the wet fungus/fungal extract with the metal salt solution is carried out at a temperature in the range of 15 to 40° C. and for a period in the range of 1 to 3 days.

3. A process as claimed in claim 1 wherein the biomass is filtered using a minimum 1 micron pore size filter to obtain the oxide nanoparticles.

4. A process as claimed in claim 1 wherein the metal'salt is selected from the group consisting of chlorides, nitrates, oxalates and sulfates.

5. A process as claimed in claim 1 wherein the fungus is used in whole cell form, wet solid mass or fungal extract.

6. A process as claimed in claim 1 wherein the temperature for incubation is in the range of 23-33° C.

7. A process as claimed in claim 6 wherein the temperature for incubation is in the range of 25-29° C.

8. A process as claimed in claim 1 wherein the concentration of the metal salt in the solution is not less than 1 mM.

9. A process as claimed in claim 1 wherein the fungus/fungal extract is used in an amount of 10 to 60 mgs.

10. A process as claimed in claim 1 wherein the fungus is selected from the group consisting of Fusarium sp., Trichothecium sp., Verticillium sp., Chloridium sp., Aspergillus sp., Cephaliophora sp., Fusarium oxysporum and Helicostylum sp.

11. A process as claimed in claim 1 wherein the metal comprises a metal from the transition metal group.

12. A process as claimed in claim 1 wherein the metal is selected from the group consisting of Ti, Zr, Si and Zn.
